(19) United States
(12) Patent Application Publication
(10) Pub. No.: US 2020/0030534 A1
(43) Pub. Date: Jan. 30, 2020

(54) MEDICAMENT INJECTION DEVICE AND METHOD

(71) Applicant: Joel Howard, Strathcona, MN (US)

(72) Inventor: Joel Howard, Strathcona, MN (US)

(21) Appl. No.: 16/048,252

(22) Filed: Jul. 28, 2018

(51) Int. Cl. A61M 5/20; A61M 5/32

(52) U.S. Cl. CPC A61M 5/2033; A61M 2005/341; A61M 5/3221; A61M 5/3234

(57) ABSTRACT

A medicament injection device including a needle, a first stage that controls positioning of the needle at a depth into an animal when the device is pressed against the animal, and a second stage that injects the medicament through the needle into the animal after the needle is positioned at the depth and the device is further pressed against the animal. The device preferably includes a mechanism that permits the second stage to inject the medicament only after the needle is positioned at the depth. The depth of the needle preferably is adjustable, as is the amount of the medicament injected. In preferred aspects, these two features are independently adjustable. One or more springs, pneumatics elements, or other elements may automatically reset the first stage, the second stage, or both after the device is withdrawn from the animal. Also, associated methods.

### BACKGROUND

The present disclosure generally relates to a medicament injections device, for example to inject vitamins or vaccines into livestock such as cattle, sheep, or the like as well as other animals.

### SUMMARY

Aspects of the subject technology include a medicament injection device including a needle, a first stage that controls positioning of the needle at a depth into an animal when the device is pressed against the animal, and a second stage that injects the medicament through the needle into the animal after the needle is positioned at the depth and the device is further pressed against the animal. The medicament may be one or more vaccines, antibiotics, or vitamins. In some aspects, the device include one or more bladders with valves that hold the medicament before injection.

The device preferably includes a mechanism that permits the second stage to inject the medicament only after the needle is positioned at the depth. The mechanism may be one or more spring-loaded locking mechanisms. Other mechanisms may be used.

The first stage preferably controls an angle of positioning the needle. The depth of the needle preferably is adjustable, as is the amount of the medicament injected. In preferred aspects, these two features are independently adjustable. One or more springs, pneumatics elements, or other elements may automatically reset the first stage, the second stage, or both after the device is withdrawn from the animal.

In some aspects, the device includes a holder for a vial of the medicament. A counter showing how many times the medicament has been injected from the vial and/or how many more time a medicament can be injected from the vial. Other elements such as an attachment for an extended handle may be included.

The subject technology also includes variations of the device discussed above, methods of using the device and variations thereof, and/or other aspects disclosed in herein.

This brief summary has been provided so that the nature of the invention may be understood quickly. Additional steps and/or different steps than those set forth in this summary may be used. A more complete understanding of the invention may be obtained by reference to the following description in connection with the attached drawings.

### BRIEF DESCRIPTION OF THE DRAWINGS

FIG. 1 illustrates a spring actuated medicament injection device and method according to aspects of the subject technology.

FIG. 2 illustrates activation of a first stage of the device shown in FIG. 1.

FIG. 3 illustrates activation of a second state of the device shown in FIG. 1.

FIG. 4 illustrates adjustment of the device shown in FIG. 1 for a deeper depth.

FIG. 5 illustrates an adjustment of an amount of medicament to be injected.

FIG. 6 illustrates another adjustment of an amount of medicament to be injected.

FIG. 7 illustrates a pneumatically actuated medicament injection device according to aspects of the subject technology.

FIG. 8 illustrates some possible additional features of a medicament injection device according to aspects of the subject technology.

FIG. 9 illustrates an attachment for an extended handle to a medicament injection device according to aspects of the subject technology.

FIG. 10 illustrates an example spring-loaded locking mechanism according to aspects of the subject technology.

### DETAILED DESCRIPTION

Briefly, techniques according to aspects of the subject technology include a medicament injection device including a needle, a first stage that controls positioning of the needle at a depth into an animal when the device is pressed against the animal, and a second stage that injects the medicament through the needle into the animal after the needle is positioned at the depth and the device is further pressed against the animal. The medicament may be one or more vaccines, antibiotics, or vitamins. In some aspects, the device include one or more bladders with valves that hold the medicament before injection.

The device preferably includes a mechanism that permits the second stage to inject the medicament only after the needle is positioned at the depth. The mechanism may be one or more spring-loaded locking mechanisms. Other mechanisms may be used.

The first stage preferably controls an angle of positioning the needle. The depth of the needle preferably is adjustable, as is the amount of the medicament injected. In preferred aspects, these two features are independently adjustable. One or more springs, pneumatics elements, or other elements may automatically reset the first stage, the second stage, or both after the device is withdrawn from the animal.

In some aspects, the device includes a holder for a vial of the medicament. A counter showing how many times the medicament has been injected from the vial and/or how many more time a medicament can be injected from the vial. Other elements such as an attachment for an extended handle may be included.

### Genesis and Overview

The following is a description of the subject technology's genesis and an overview thereof in the inventor's own words. Minor grammar corrections, changes to clarify some aspects, and some other editorial changes have been made.

Last summer I had a cow that got loose into the open pasture before calving and she was so mean when she finally calved that I couldn't get close to her or her calf to administer shots. There are clostridial pathogens that are present in the pasture and for a new calf it can cause it to get behind and get sick. All too often treatment after the fact is a lost cause. I could not get her and the calf even into the pasture with a corral. I tried setting up gates and coaxing her with feed. I could get within arms-length, but that was it. I even built a pen that attached to the loader tractor in an attempt to pen her up with that. I could always get a couple of feet from her. I had an idea!

I took a syringe assembly that I had and disassembled it. I cut the steel that separated the handle from the plunger. I got a six-foot chunk of PVC. Found a six foot chunk of ¼″ round steel and welded it in between the plunger and the handle making myself a 6′ long syringe. Pain in the butt to load it cause it's 6 feet long, but I loaded it up, walked out to the pasture to the cow and calf, and proceeded to give them both shots from 4 to 6 feet away. It worked for the task at hand but all these thoughts of what I could do to make this better occupied my mind.

This winter while vaccinating calves and getting ready for weaning it became apparent that there lies a much easier way to work cattle and give shots. Something that can be faster, more efficient and safer.

Normal routine is place a single animal in a chute and give it 3 to 4 different shots. Each one of the shots is reloaded into a single dose syringe or an automatic repeater syringe and administered by using your fingers or palm and because there is a control factor that needs to take place, you have to put your other hand in the chute system to make sure that the shot isn't given too deep or too shallow. Everything about it screams time! And when you are doing it by yourself it can be a two-day affair for one person working 100 pairs.

So how do we improve this?

Stick to automatic reloading and dispensing syringe. I need something that can administer the shot by simply jabbing the animal and not have to touch it every time. In order to do this I need to ensure that the needle entry point can be the same all the time every time. A piston on the end of the syringe that can act as a flesh displacing print to level the shot location. This piston will also act as the trigger that once pushed inside releases the liquid and is dispensed through the needle. It will also act as a protective shroud that hides the needle until administration of the vaccine is required. This action can be mechanical or something like air can be used.

The action piston not only protects the needle, but it also can protect the user from the needle. Big safety advantage. Also it keeps it contained as to prevent damage or external contamination of the needle. Because the needle is contained inside the mechanism there is the problem of cross contamination between animals. Meaning blood and genetic material can be concealed along with the needle. On the farm we simply dip the syringe in soapy water and it helps. I propose an exhaust port in the applicators external piston that allows you to quickly spray it between use washing the needle off directly while keeping everything else contained and out of contamination.

The dosage as well as the depth of penetration should be adjustable. The dosage adjustment could be as simple as sleeving the chamber with another chamber that it can thread in elongating or compressing the chamber and the piston will work right along with this. The dosage should be adjustable to half a cc. For example 0.5 cc to 1 cc. Eventually perhaps smaller versions could be made for swine, dogs and cats. The penetration could be handled by being able to adjust the external piston in or out. Perhaps threaded or some form of notching system as threading could be problematic allowing the piston to move on its own?

The vaccine port will have to be able to hold safely in place a 10-dose bottle to as large as a 50-dose bottle. I think that the best way to achieve this is to have a canister or containment area that the bottles can be put into and secured as to not be disrupted from their location. A retaining spring and perhaps a closed canister.

The handle or how the device is held is another thing to explore. Adding a handle on the rear end of the device keeps it streamlined and goes along with the safety theme of it. Keeping the users hands clear and allowing application to be done from distances. Perhaps a telescoping handle.

Also needed is a way to stop it from drawing or reloading itself. For example if you have only two animals left to vaccinate, but you are loaded for 10, you could simply close the vaccine bottle valve to not draw after the last shot is administered.

Adding color to the devices is also something that could be done. I would like to see the products produced by machined parts. So perhaps the main structure could be anodized or powder coated. The reason for this, is that the customer will seemingly purchase 2 to 4 of these and if they are colored, they can designate the color for a certain vaccine use and dedicate it to such.

### Other Applications

This device isn't limited to just cattle. It could be incorporated and sized to work in a multitude of situations. Any livestock setting. zoo's and zookeepers could find this useful from a safety standpoint. Dog kennels or pounds. Genetic dispensing and circumstances where repetitive dosage and administration settings are required.

Other positives that may come from this device:
Shots can be given from distance.
Shots can be administered in a multitude of settings.
Does not require chute systems or head gates.
Theoretically the device could be thrown and the impact would administer the dosage.
Hands free operation.
Needle protection for the device and the user.
Adjustability.

The device may help take the guess work out of the equation for the applicator. When having to push a plunger or squeeze a trigger or handle you can never be certain that it is happening exactly when you desire. Brain lag is real. Fatigue of mind. When the device reaches the parameters set it administers the drug. Consistency!

Some form of shot counter should be incorporated. I cannot tell how many times something has happened, and you get sidetracked from working the cows. You cannot remember if you gave a certain shot to a cow, so you give another one to be certain. Also this will let you know where you are at with your vaccines.

The subject technology is not limited to the Genesis and Overview discussed above, which is provided to explain some real-world aspects of the technology from the perspective of a person who has extensive experience dealing with livestock.

### Application to Humans

Unfortunately, opioid abuse is a common problem. A person suffering from an overdose of an opioid such as heroine can be violently resistant to treatment with a medicament such as Naloxone. About 4 to 5 cc's of Naloxone typically is sufficient to neutralize opioids in a person. However, even though the person may be dying from the overdose, they often violently resist administration of Naloxone or the like.

Other drug abuses are also unfortunately common and potentially dangerous to first responders. For example, a person under the influence of phencyclidine (PCP) is capable of nearly inhuman feats of strength, for example fighting off multiple first responder even while literally breaking their own bones and/or dislocating their joints.

Some people also suffer from behavioral disorders that can result in violence, nearly inhuman feats of strength, and the like. Encounters with such people by first responders can be dangerous both for the person with the disorder and the responder(s).

Aspects of the subject technology my permit first responders to administer appropriate medicaments such as Naloxone, tranquilizers, or the like while maintaining a safe distance from the person under influence of opioids, PCP, other drugs including narcotics, suffering from behavioral disorders, and the like.

### Illustrative Embodiments and Aspects

Possible embodiments and aspects of the subject technology are discussed below with respect to the accompanying figures. The technology is not limited to these embodiments and aspects.

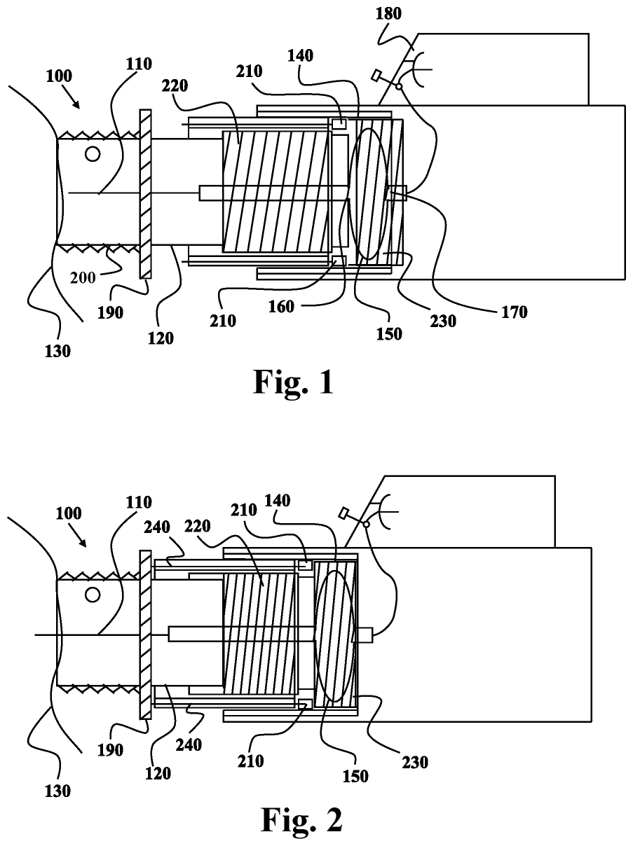

FIG. 1 illustrates a spring actuated medicament injection device and method according to aspects of the subject technology. The medicament may be or include one or more vaccines, antibiotics, or vitamins.

Device 100 preferably includes needle 110, first stage 120, and second stage 130. First stage 120 may control positioning of the needle at a depth into animal 130 when the device is pressed against the animal. The stage may take the form of a piston, edges of which align flesh of animal 130 for properly angled injection when pressed against the animal.

Second stage 140 may also take the form of a piston. The second stage preferably injects the medicament through the needle into the animal after the needle is positioned at the proper depth and the device is further pressed against the animal.

Bladder 150 that holds the medicament before injection preferably is included in and/or works in conjunction with second stage 140. The bladder preferably is made of a suitably elastic material such as but not limited to rubber, plastic, polyurethane, or the like. Additional bladders may be included, as discussed below.

The bladder preferably includes valves 160 and 170. In the illustrated aspects, valve 160 permits injection of the medicament from bladder 150 when the second stage is activated, and valve 170 permits reloading of bladder 150 from a vial of medicament connected by holder/attachment 180 when the device is withdrawn from an animal.

The depth of needle penetration when device 100 is pressed against an animal preferably is adjustable. For example, a position of ring 190 on threads 200 may be adjusted, thereby controlling a depth of needle penetration.

In preferred aspects, mechanism(s) 210 permits the second stage to inject the medicament only after the needle is positioned and inserted at the chosen depth. An example of such mechanism(s) is a spring-loaded locking mechanism, for example of the type akin to that used with locking knives, firearm safeties, and/or auto-locking doors. One example of such a mechanism is discussed with respect to FIG. 10. Other mechanisms may be used.

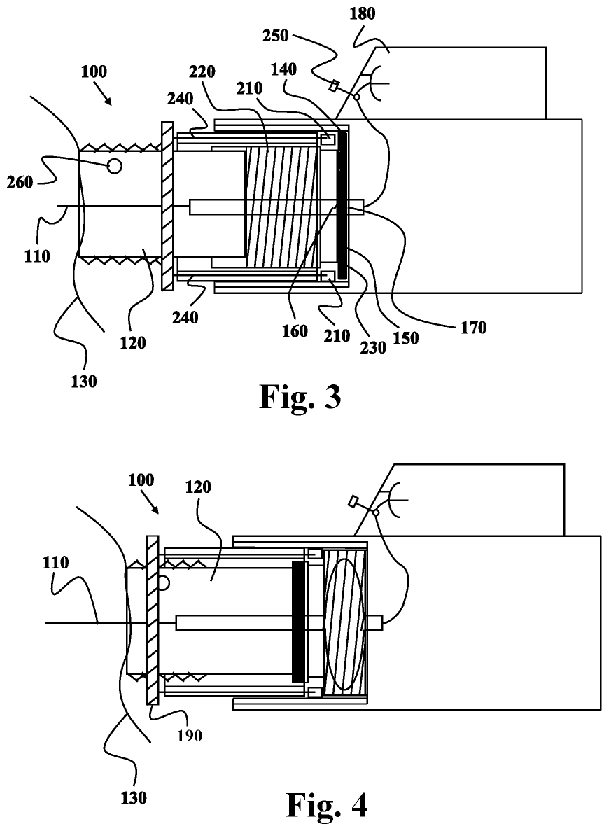

In FIG. 1, spring 220 resets first stage 120 and spring 230 resets second stage 140 after injection has occurred and device 100 is withdrawn from animal 130. FIGS. 2 and 3 further illustrate an example the injection process.

Briefly, device 100 is pressed against an animal. The first stage in some aspects controls positioning of the needle at a depth into the animal when the device is pressed against the animal. The second stage in some aspects injects the medicament into the animal through the needle after the needle is positioned at the depth and the device is further pressed against the animal. The device can then be withdrawn from the animal, preferably causing the stages to reset automatically.

FIG. 2 illustrates activation of first stage 120 of device 100. The device is shown pressed against animal 130, activating stage 120 to expose needle 110. The needle preferably penetrates the animal at a depth set by ring 190. Spring 220 is shown as partially compressed. Mechanism(s) 210 have prevented activation of second stage 140 until activation of the first stage is complete. Thus, bladder 150 and spring 230 are shown as not being compressed. Note that in some circumstances, partial activation of second stage 140 may occur before first stage 120 has been fully activated, although such is not preferred.

In FIG. 2, ring 190 is shown pressing against pin(s) 240, activating mechanism(s) 210. This process preferably allows second stage 140 to be activated.

FIG. 3 illustrates activation of device 100's second stage 140. Continued pressure against animal 130 results in compression of bladder 150 and associated spring 230. Compression of bladder 150 creates pressure in the bladder that opens valve 160, permitting injection of the medicament into animal 130 via needle 110. The pressure also closes valve 170, lessening a likelihood of contamination of a medicament vial held by holder/attachment 180.

After injection of the medicament is complete, device 110 preferably is withdrawn. Springs 220 and 230 then automatically reset first stage 120 and/or second stage 140. Resetting second stage 140 preferably draws an additional dose of medicament from the vial held by holder/attachment 180 through valve 170. Resetting first stage 120 also preferably re-engages mechanism(s) 210 by releasing pressure from pin(s) 240. Thus, device 100 is ready to be used to inject medicament into another animal.

FIG. 3 also illustrates valve/control 250 that may be used to cut off supply of medicament from the vial held by holder/attachment 180. For example, if a last animal is about to be injected with medicament, the valve/control may be used to shut off the supply so as not to waste medicament.

Also shown is port 260 that preferably is provided to permit cleaning of needle 110 and an interior of at least part of first stage 120 between injections, for example via flushing with saline or other cleaning fluids. The port preferably does not communicate with bladder 150 due to valve 160 being closed when injection is not occurring. Thus, a potential of contaminating the bladder during cleaning should be reduced.

FIG. 4 illustrates adjustment of device 100 for a deeper depth of needle penetration. In this figure, ring 190 has been adjusted toward an end of first stage 120 closer to animal 130. Thus, more of needle 110 is exposed when the first stage is activated.

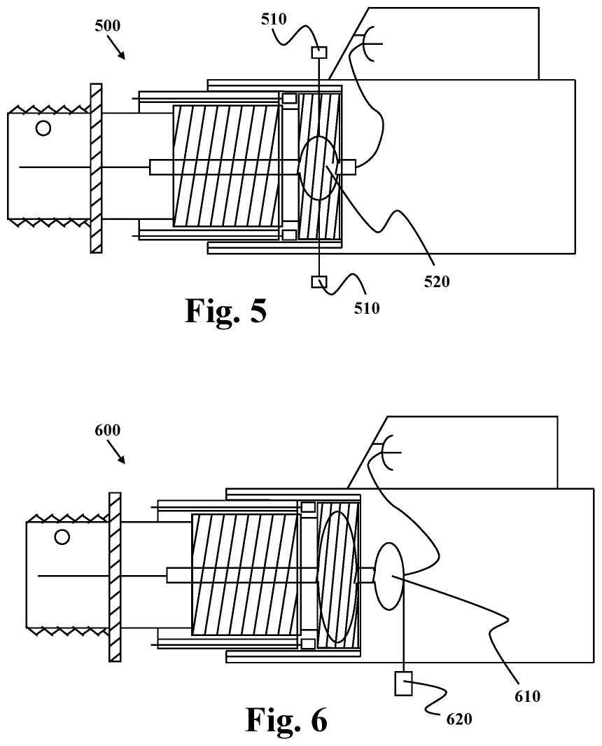

FIG. 5 illustrates an adjustment of an amount of medicament to be injected. Device 500 includes control(s) 510 to adjust a size of bladder 520, which otherwise is akin to bladder 150 discussed above.

FIG. 6 illustrates another adjustment of an amount of medicament to be injected. Device 600 includes a secondary bladder 610, the size of which may be adjusted using control 620. Other mechanisms for adjusting the amount of medicament injected with each use may also be incorporated instead of or in addition to those shown in FIGS. 5 and 6.

In preferred aspects, the depth of injection and the amount of medicament injected may be independently controlled. For example, as shown in the figures, ring 190 and control(s) 510 and 620 may be independently adjusted. Other approaches to achieving such independent adjustment are within the scope of the subject technology.

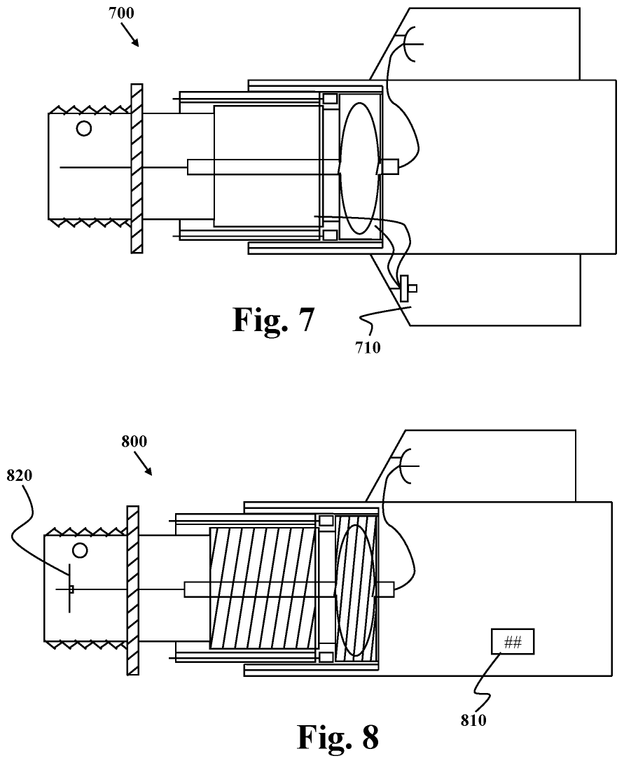

FIG. 7 illustrates a pneumatically actuated medicament injection device according to aspects of the subject technology. While FIGS. 1 to 6 illustrate use of springs, other mechanism(s) for resetting and otherwise controlling operation of the devices may be used. In one example shown in FIG. 7, holder 710 is provided for a pneumatic system or element such as a CO2 cartridge that may be used to automatically reset the first stage, the second stage, or both after device 700 is withdrawn from an animal.

FIG. 8 illustrates some possible additional features of a medicament injection device according to aspects of the subject technology. One such feature is counter 810 that shows how many times the medicament has been injected from a vial and/or how many more time a medicament can be injected from the vial using device 800. Basic mechanical and/or electronic elements may be included to control the counter. Another such feature is a preferably sliding plate 820 on a needle of the device intended to help ensure proper alignment of the needle when penetrating an animal.

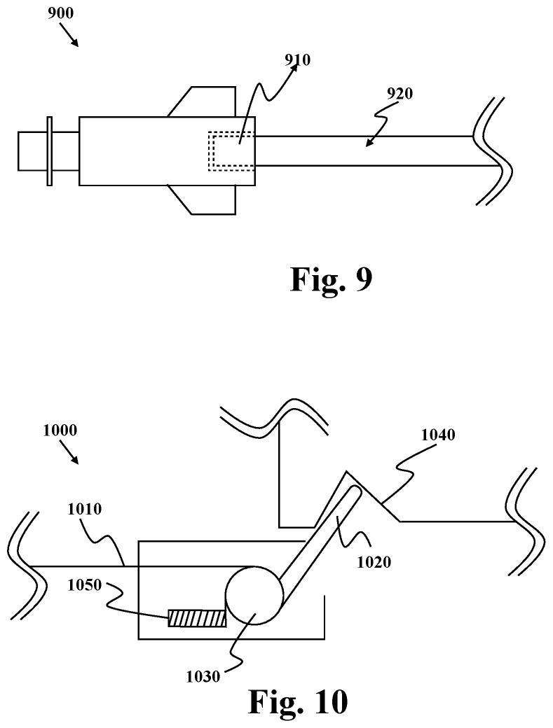

FIG. 9 illustrates attachment 910 for an extended handle 920 to medicament injection device 900 according to aspects of the subject technology. The attachment is illustrated as a socket. Any other form of attachment may be used. The extended handle may for example be a telescoping handle.

FIG. 10 illustrates an example spring-loaded locking mechanism according to aspects of the subject technology. Mechanism 1000 is activated by pressure from pin 1010. Pressure from the pin causes latch 1020 to rotate around pivot 1030. Before rotation, latch 1020 engages slot 1040, preventing movement of the mechanism. After rotation, latch 1020 clears slot 1040, allowing movement of the mechanism. When pressure from spring 1010 is absent and the locking mechanism returns, spring 1050 re-engages latch 1020 with slot 1040. Other locking mechanisms of various designs may be used with aspects of the subject technology.

All components of the devices and aspects herein discussed preferably are made of suitably durable, weather resistant, and otherwise appropriate materials. In addition, bladders, springs, and the like preferably are made of materials suitable for repeated use. The devices also preferably are easily opened to permit access for replacing parts that may fatigue after use.

The invention is in no way limited to the specifics of any particular aspects (e.g., embodiments, elements, steps, and/or examples) disclosed herein. For example, the terms “aspect,” “example,” “preferably,” “may,” “alternatively,” and the like denote features that may be preferable but not essential to include in some embodiments of the invention. Details illustrated or disclosed with respect to any one aspect of the invention may be used with other aspects of the invention. Additional elements and/or steps may be added to various aspects of the invention and/or some disclosed elements and/or steps may be subtracted from various aspects of the invention without departing from the scope of the invention. Singular elements/steps imply plural elements/steps and vice versa. Some steps may be performed serially, in parallel, or in different orders than disclosed herein. Many other variations are possible which remain within the content, scope, and spirit of the invention, and these variations would become clear to those skilled in the art after perusal of this application.

What is claimed is:

1. A medicament injection device, comprising:
a needle;
a first stage that controls positioning of the needle at a depth into an animal when the device is pressed against the animal; and
a second stage that injects the medicament through the needle into the animal after the needle is positioned at the depth and the device is further pressed against the animal.

2. The medicament injection device of claim 1, wherein the medicament comprises one or more vaccines, antibiotics, or vitamins.

3. The medicament injection device of claim 1, further comprising one or more bladders with valves that hold the medicament before injection.

4. The medicament injection device of claim 1, further comprising a mechanism that permits the second stage to inject the medicament only after the needle is positioned at the depth.

5. The medicament injection device of claim 4, wherein the mechanism comprises one or more spring-loaded locking mechanisms.

6. The medicament injection device of claim 1, wherein the first stage also controls an angle of positioning the needle.

7. The medicament injection device of claim 1, wherein the depth is adjustable.

8. The medicament injection device of claim 1, wherein an amount of the medicament injected is adjustable.

9. The medicament injection device of claim 1, the depth and an amount of the medicament injected are independently adjustable.

10. The medicament injection device of claim 1, further comprising one or more springs that automatically reset the first stage, the second stage, or both after the device is withdrawn from the animal.

11. The medicament injection device of claim 1, further comprising one or more pneumatic elements that automatically reset the first stage, the second stage, or both after the device is withdrawn from the animal.

12. The medicament injection device of claim 1, further comprising a holder for a vial of the medicament.

13. The medicament injection device of claim 12, further comprising a counter showing how many times the medicament has been injected from the vial.

14. The medicament injection device of claim 12, further comprising a counter showing how many more time a medicament can be injected from the vial.

15. The medicament injection device of claim 1, further comprising an attachment for an extended handle.

16. A medicament injection method, comprising:
pressing a device against an animal, the device including a needle, a first stage that controls positioning of the needle at a depth into the animal when the device is pressed against the animal, and a second stage that injects the medicament into the animal through the needle after the needle is positioned at the depth and the device is further pressed against the animal; and
withdrawing the device from the animal.

17. The medicament injection method of claim 16, wherein the medicament comprises one or more vaccines, antibiotics, or vitamins.

18. The medicament injection method of claim 16, further comprising independently adjusting the depth and an amount of the medicament injected into the animal.

19. The medicament injection method of claim 16, wherein the first stage, the second stage, or both are spring actuated to automatically reset after withdrawal of the device from the animal.

20. The medicament injection method of claim 16, wherein the first stage, the second stage, or both are pneumatically actuated to automatically reset after withdrawal of the device from the animal.
